(19) United States
(12) Patent Application Publication
(10) Pub. No.: US 2004/0147430 A1
(43) Pub. Date: Jul. 29, 2004

(54) Use of antidiabetics for making a medicine with cicatrizing effect

(76) Inventor: Philippe Briet, Lyon (FR)

Correspondence Address: Young & Thompson, Second Floor, 745 South 23rd Street, Arlington, VA, 22202, US

(21) Appl. No.: 10/479,615

(22) PCT Filed: Jun. 19, 2002
(86) PCT No.: PCT/FR02/02124

(30) Foreign Application Priority Data
Jun. 2, 2001 (FR) 01 08130

(51) Int. Cl. A61K 38/28; A61K 31/4439; A61K 31/426; A61K 31/175

(52) U.S. Cl. 514/3; 514/12; 514/342; 514/369; 514/592

(57) ABSTRACT

The invention concerns the use of at least an antidiabetic selected among compounds stimulation insulin secretion, glucosidase inhibitors, thiazolidine-diones, insulin, agents enhancing insulin sensitivity, the glucagon-like peptide-1 (GLP-1), PPARα/γ agonists, meglitinide and aP2 inhibitors for making a medicine with cicatrizing effect.

The present invention relates to the cicatrization of wounds.

It relates in particular to the use of antidiabetics for the manufacture of a medicine having a cicatrizing effect.

The cicatrization of wounds or of related injuries of various types of tissue generally depends on the proliferation of new epithelial, endothelial and connective tissues. It therefore calls into play a series of coordinated, cellular and molecular events. It can be delayed or modified by metabolic disturbances which accompany certain diseases of long duration such as venous insufficiency, arthritis, diabetes and even by certain therapies.

The pharmaceutical market currently offers numerous topical preparations which are recommended for the cicatrization of wounds. In fact, their action results from the complementarity of various products which constitute them and which confer on them within a certain limit their cicatrizing property. They protect the wounds from the surrounding environment with an antiseptic dressing. They stimulate the development of vascularization and regulate epidermalization. These topical forms consist mainly of a lipid mixture (lanolin, petroleum jelly, glycerin, and the like) to which acids (salicylic, benzoic, malic), minerals (zinc oxide, titanium oxide) or halides (starch iodide) are added.

Some also contain collagen, fibrinogen, enzymatic proteolysates of serum (supply of amino acids) or vitamins (vitamin A) or hormones (4-chlorotestosterone acetate). An ointment (Madecasol® tulgras from acetate). An ointment (Madécasol® tulgras from Laboratoires SYNTEX) also exists whose cicatrizing action is provided by the addition of a mixture of three triterpenes extracted from roots of the plant Centella asiatica (TCEA). These compounds exert their property by stimulating the biosynthesis of collagen and of glycoaminoglycans. However, these extracts can also cause contact allergies in patients.

It is known that one of the complications of diabetes consists in the appearance of skin conditions such as ulcers (see ulcerative necrotic angiodermatitis) or perforating dermatoses which the conventional medicines used during the treatments of diabetes do not succeed either in controlling or in treating.

Pharmaceutical compositions based on antidiabetics are thus already known. However, they are not known to have a cicatrizing effect.

Now, the inventors of the present invention have surprisingly demonstrated that some antidiabetics also possess strong cicatrizing properties, that is to say a stimulatory activity in relation to a complex physiological phenomenon characterized inter alia by increased cellular growth at the site of the wound. This transient proliferation occurs in response to the loss of skin integrity and brings about the repair of deep tissue and the reconstitution of the epidermis at the site of the wounds.

Thus, the topical application of one of these compounds in the form of an ointment induces a rapid and lasting healing of leg ulcers in diabetic subjects and the repetition of the topical application of the active ingredient reinforces this effect. Furthermore, these antidiabetics can also accelerate the cicatrization of atonic wounds in nondiabetic subjects.

Taking into account the difficulties encountered in bringing under control the quality of natural cicatrizing products and the number of tedious steps necessary for isolating these compounds, oral antidiabetics, whose synthesis is in general simple, total and rapid, appear as very advantageous active ingredients.

The present invention therefore relates to the use of at least one antidiabetic chosen from compounds stimulating the secretion of insulin, glucosidase inhibitors, thiazolidinediones, insulin, agents enhancing sensitivity to insulin, glucagon-like peptide-l (GLP-1), PPAR α/γ agonists, meglitinide and aP2 inhibitors, for manufacturing a medicine having a cicatrizing effect.

Preferably, said medicine is in a form for topical use. Advantageously, this medicine is intended to be applied to the skin.

These antidiabetics may be used alone or in combination.

As compounds stimulating the secretion of insulin, there may be mentioned sulfonylureas. As examples of sulfonylureas, there may be mentioned in particular acetohexamide, carbutamide, gliquidone, glisentide, glisolamide, glisoxepide, glycyclamide, glibornuride, chlorpropamide, tolazamide, tolbutamide, tolcyclamide, glipizide, gliclazide, glimepiride and glibenclamide.

There may also be mentioned, as compounds stimulating the secretion of insulin by the β cells of the pancreas, repaglinide, nateglinide or nitiglinide.

As examples of glucosidase inhibitors and more particularly α-glucosidase inhibitors, there may be mentioned acarbose (described in patent U.S. Pat. No. 4,904,769), vaglibose and miglitol (described in patent U.S. Pat. No. 4,639,436).

Among the thiazolidinediones or the other agents promoting sensitivity to insulin (which have a sensitizing effect to insulin in patients having noninsulin-dependent diabetes), there may be mentioned troglitazone (Rezuline® from the company Warner Lambert, described in patent U.S. Pat No. 4,572,912), Rosiglitazone (SKB-Avandia®), Pioglitazone (Actos® from the company Takeda-Lilly), MCC-555 from the company Mitsubishi (described in patent U.S. Pat No. 5,594,016), GL-262570 from the company Glaxo-Welcome, englitazone (CP-68722 from the company Pfizer) or darglitazone (CP-86325 from the company Pfizer).

There may also be mentioned, as thiazolidinedione, the compound of formula (I) defined in the following manner:

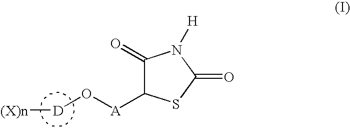

in which A represents a linear or branched, saturated or unsaturated hydrocarbon group comprising from 2 to 16 carbon atoms,

D represents a homo- or heterocarbon, mono, bi- or tricyclic aromatic structure which may include one or more heteroatoms,

X represents a substituent of the aromatic structure, selected from hydrogen, an alkyl group having from 1 to 6 carbon atoms, an alkoxy group having from 1 to 6 carbon atoms, an alkoxyalkyl group in which the alkoxy and alkyl groups are defined as above, an aryl group defined as an aromatic cyclic structure containing one or two rings optionally including one or two heteroatoms in the ring such as for example a phenyl or an α- or β-naphthyl, an arylalkyl group in which the alkyl group is defined as above and the aryl group is defined as above and optionally contains one or more substituents, an arylalkylaryl group whose arylalkyl and aryl fractions are defined as above, a halogen, a trifluoromethyl, a cyano, a hydroxyl, a nitro, an amino, a carboxyl, an alkoxycarbonyl, a carboxamide, a sulfonyl, a sulfone, a sulfonamide, a sulfamoyl, an alkylsulfonylamino, an acylamino, a trifluoromethoxy,

n is an integer ranging from 1 to 3,

with the restriction that if A represents the butyl radical,

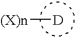

does not represent the 4-chlorophenyl group.

In the preceding text, among the aromatic radicals D, there may be mentioned as homocarbon structure the phenyl, α-naphthyl, β-naphthyl, anthracene or fluorenyl radical. Among the heterocyclic aromatic radicals, there may be mentioned pyridyl, the quinolinyl or carbazolyl ring.

D preferably represents the phenyl or naphthyl radical.

Among the alkyl groups having from 1 to 6 carbon atoms, there may be mentioned in particular a methyl, ethyl, propyl, isopropyl, butyl, isobutyl, tert-butyl, pentyl or hexyl radical. Among the alkoxy groups having from 1 to 6 carbon atoms, there may be mentioned in particular a methoxy, ethoxy, propoxy, isopropoxy, butoxy or isobutoxy radical. Among the halogen groups, there may be mentioned in particular fluorine, chlorine, bromine or iodine.

The chain A is a hydrocarbon chain having from 2 to 16 carbon atoms, which is linear or branched, saturated or once or several times ethylenic, optionally substituted with at least one hydroxyl radical or with a phenyl radical. As examples of a linear alkyl radical, there may be mentioned in particular a divalent ethyl, propyl, butyl, pentyl, hexyl, octyl, nonyl, decyl, dodecyl or hexadecyl radical. Among the branched alkyl chains, there may be mentioned in particular the divalent 2-ethylhexyl, 2-methylbutyl, 2-methylpentyl, 1-methylhexyl or 3-methylheptyl radical. Among the monohydroxyalkyl chains, the radicals having 2 or 3 carbon atoms, such as 2-hydroxyethyl, 2-hydroxypropyl or 3-hydroxypropyl, are preferred. Among the polyhydroxyalkyl chains, the radicals having 3 to 6 carbon atoms and 2 to 5 hydroxyl radicals, such as 2,3-dihydroxypropyl, 2,3,4-trihydroxybutyl or 2,3,4,5-tetrahydroxypentyl, or the pentaerythritol residue are preferred. Among the hydrocarbon chains having from 2 to 16 carbon atoms and one or more times ethylenic, there may be mentioned in particular the divalent allyl radical.

The divalent ethyl or propyl radical is preferred.

The present invention also relates to the tautomeric forms of the compounds of general formula (I) , to the enantiomers, diastereoisomers and epimers of these compounds, and to their solvates.

It can be conceived that the ketone functional groups carried by the thiazolidine ring can become enolized and give rise to monoenols.

The thiazolidinedione derivatives may in this case be salified and may exist in the form of basic salts.

Examples of basic salts of the compounds of general formula (I) include pharmacologically acceptable salts such as sodium salts, potassium salts, magnesium salts, calcium salts, amine salts and other salts of the same type (aluminum, iron, bismuth, and the like). The amine salts which are not pharmacologically acceptable can serve as a means of identification, purification or resolution.

Among the compounds of general formula (I), there may be mentioned more particularly as currently preferred compounds:

5-[3-(4-fluorophenoxy)propyl]thiazolidine-2,4-dione

5-(2-phenoxyethyl)thiazolidine-2,4-dione

5-[2-(4-fluorophenoxy)ethyl]thiazolidine-2,4-dione

5-{[1-hydroxy-2-(4-fluorophenoxy)]ethyl}thiazolidine-2,4-dione

5-{[2-hydroxy-3-(4-fluorophenoxy)]propyl}thiazolidine-2,4-dione

5-[l-methyl-2-phenoxyethyl]thiazolidine-2,4-dione

5-[2-(4-cyanophenoxy)ethyl)]thiazolidine-2,4-dione

5-[2-(2-fluorophenoxy)ethyl]thiazolidine-2,4-dione

5-[2-(2-naphthyloxy)ethyl]thiazolidine-2,4-dione and their pharmacologically acceptable salts.

These compounds have been described in patent application WO 97/47612.

The use of 5-[2-(4-cyanophenoxy)ethyl)]thiazolidine-2,4-dione is preferred.

The aP2 inhibitors may be selected in particular from the compounds described in patent WO 200059506, WO 200015230 and WO 200015229, filed by the company Bristol-Myers Squib.

Among the nonoral antihyperglycemic agents, there may therefore be mentioned insulin or glucagon-like peptide-1 (GLP-1), such as GLP-1(1-36) amide, GLP-1(7-36) amide, GLP-1(7-37), as described in patent U.S. Pat. No. 5,614,492.

Among the PPAR α/γ agonists, there may be mentioned in particular the N-benzyldioxothiazolidylbenzamide derivatives described in patent application WO 96/38428, such as 5-(2,4-dioxothiazolidin-5-ylmethyl)-2-methoxy-N-[4-(trifluoromethyl)benzyl]benzamide (KRP-297), in patent application WO 98/05531 (Ligand Pharmaceuticals Inc.) which discloses 2-(4-[2,4-difluorophenyl]-1-heptylureido)ethyl]phenoxy)-2-methylbutyric acid, in patent applications WO 97/25042 and WO 96/04260 (SKB) which describe benzoxazole and pyridine derivatives of structure (II)

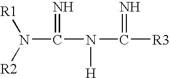

or one of its pharmaceutically acceptable salts or solvates in which R° represents 2-benzoxazolyl or 2-pyridyl and R1 represents CH2OCH3 or CF3, such as (S)-3-(4-[2-[N-(2-benzoxazolyl)-N-methylamino)ethoxy]phenyl]-2-(2-methoxyethoxy)propanoic acid or (S)-3-[4-[2-[N-(2-benzoxazolyl)-N-methylamino]ethoxy]phenyl]-2-(2,2,2-trifluoroethoxy)propanoic acid.

The term “pharmaceutically acceptable salt” is understood to mean, for the purpose of the present invention, any salt prepared from any pharmaceutically acceptable nontoxic acid, including organic and inorganic acids. Such acids include acetic, benzenesulfonic, benzoic, citric, ethanesulfonic, fumaric, gluconic, glutamic, hydrobromic, hydrochloric, lactic, maleic, malic, mandelic, methanesulfonic, mucic, nitric, pamoic, pantothenic, phosphoric, succinic, tartaric and paratoluenesulfonic acid. Advantageously, hydrochloric acid is used.

The term “pharmaceutical dosage form for topical use” is understood to mean, for the purposes of the present invention, any pharmaceutical dosage form intended to be applied to the surface of the wound, in particular to the skin or the external or internal mucous membranes, and which acts locally. In particular, the medicine may be provided in a form of the oil, cream, foam, liniment, lotion, ointment, liquid, gel, milk, powder or spray type. The forms may contain a monophasic vehicle, consisting of a neutral gel of hydroxypropylcellulose or of a charged gel consisting of sodium carboxymethylcellulose. It is also possible to prepare creams, forms with a biphasic vehicle, containing a hydrophilic phase dispersed in a lipophilic phase. Preferably, the medicament is not in the form of a polymer-based dressing.

In a particular example, the medicament contains from 0.02 to 2% by weight of one of these antibiotic agents or of its pharmaceutically acceptable salt and an appropriate excipient.

These excipients may be chosen from compounds having good compatibility with these active ingredients. They are for example water-soluble polymers of the natural polymer type, such as polysaccharides (xanthan gum, carob gum, peptin, and the like) or polypeptides, cellulose derivatives of the methylcellulose, hydroxypropylcellulose, hydroxypropylmethylcellulose type or alternatively synthetic polymers, polaxamers, carbomers, PVA or PVP.

Finally, it is within the capability of persons skilled in the art to add to these medicines various excipients, for example cosolvents such as ethanol, glycerol, benzyl alcohol, humectants (glycerol), diffusion-facilitating agents (transcurol, urea), or alternatively antibacterial preservatives (methyl p-hydroxybenzoate at 0.15%).

In a particular embodiment of the invention, the antidiabetics used in the present invention are combined with at least one other active ingredient. The latter may be for example another antibiotic agent such as one of those described above or another antifungal or antiviral agent, which makes it possible to accelerate the cicatrization of inflammations or damaged and infected tissues, simultaneously or in coordination with the treatment of the underlying infection.

This active ingredient may also constitute another cicatrization-improving agent such as for example epidermal growth factor, fibroblast growth factor, platelet derived growth factor, and the like.

The antidiabetics used in the present invention can therefore improve the cicatrization of any type of wounds or lesions. These wounds or lesions may be of the type including surgical incisions, thermal burns, chemical burns or burns caused by irradiation, abrasions, lacerations, amputations, ischemic ulcers or decubitous ulcers, mouth lesions or ulcers or lesions of the cornea, and in particular those caused by a surgical intervention carried out in weakened subjects, elderly subjects, subjects treated by radio- or chemotherapy, or diabetics. The same applies to all the dermatoses observed in patients whose skin circulation is deficient (erythematous lesions, vasculitis) and all the wounds observed in diabetic subjects. The pharmaceutical compositions and medicines according to the invention also appear to be beneficial in the therapy of post-thrombotic, tissue necrosis for example.

1. The use of at least one antidiabetic chosen from compounds stimulating the secretion of insulin, glucosidase inhibitors, thiazolidinediones, insulin, agents enhancing sensitivity to insulin, glucagon-like peptide-1 (GLP-1), PPAR α/γ agonists, meglitinide and aP2 inhibitors, for manufacturing a medicine having a cicatrizing effect.

2. The use as claimed in claim 1, for manufacturing a medicine having a cicatrizing effect in a pharmaceutical dosage form for topical use.

3. The use as claimed in claim 2, for manufacturing a medicine having a cicatrizing effect in a pharmaceutical dosage form for topical use intended to be applied to the skin.

4. The use as claimed in any one of the preceding claims, for manufacturing a medicine having a cicatrizing effect on the wounds of diabetic subjects.

5. The use as claimed in any one of the preceding claims, characterized in that the compounds stimulating the secretion of insulin are selected from sulfonylureas and repaglinide, nateglinide or nitiglinide.

6. The use as claimed in the preceding claim, characterized in that the sulfonylureas are selected from acetohexamide, carbutamide, gliquidone, glisentide, glisolamide, glisoxepide, glycyclamide, glibornuride, chlorpropamide, tolazamide, tolbutamide, tolcyclamide, glipizide, gliclazide, glimepiride and glibenclamide.

7. The use as claimed in one of claims 1 to 4, characterized in that the glucosidase inhibitor is chosen from acarbose, vaglibose and miglitol.

8. The use as claimed in one of claims 1 to 4, characterized in that the thiazolidinediones and the other agents promoting the sensitivity to insulin are chosen from troglitazone, Rosiglitazone, Pioglitazone, MCC-555, GL-262570, englitazone and darglitazone.

9. The use as claimed in one of claims 1 to 4, characterized in that the thiazolidinedione is a compound of formula (I) defined in the following manner: 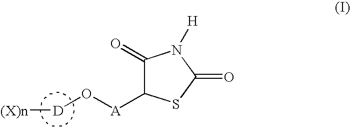
in which A represents a linear or branched, saturated or unsaturated hydrocarbon group comprising from 2 to 16 carbon atoms,
D represents a homo- or heterocarbon, mono, bi- or tricyclic aromatic structure which may include one or more heteroatoms,
X represents a substituent of the aromatic structure, selected from hydrogen, an alkyl group having from 1 to 6 carbon atoms, an alkoxy group having from 1 to 6 carbon atoms, an alkoxyalkyl group in which the alkoxy and alkyl groups are defined as above, an aryl group defined as an aromatic cyclic structure containing one or two rings optionally including one or two heteroatoms in the ring such as for example a phenyl or an α- or β-naphthyl, an arylalkyl group in which the alkyl group is defined as above and the aryl group is defined as above and optionally contains one or more substituents, an arylalkylaryl group whose arylalkyl and aryl fractions are defined as above, a halogen, a trifluoromethyl, a cyano, a hydroxyl, a nitro, an amino, a carboxyl, an alkoxycarbonyl, a carboxamide, a sulfonyl, a sulfone, a sulfonamide, a sulfamoyl, an alkylsulfonylamino, an acylamino, a trifluoromethoxy,
n is an integer ranging from 1 to 3,
with the restriction that if A represents the butyl radical, 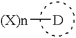
does not represent the 4-chlorophenyl group.

10. The use as claimed in one of claims 1 to 4, characterized in that the PPAR α/γ agonists are N-benzyldioxothiazolidylbenzamide derivatives.

11. The use as claimed in any one of the preceding claims, characterized in that the antidiabetic defined according to one of the preceding claims is combined with at least one other active ingredient.
